(19) United States
(12) Patent Application Publication
(10) Pub. No.: US 2006/0134211 A1
(43) Pub. Date: Jun. 22, 2006

(54) Multi-layer drug delivery device and method of manufacturing same

(75) Inventors: Mao-Jung Maurice Lien, Maple Ridge (CA); Doug Smith, Vancouver (CA); Dean-Mo Liu, Richmond (CA)

Correspondence Address: OYEN, WIGGS, GREEN & MUTALA LLP;480 - THE STATION, 601 WEST CORDOVA STREET, VANCOUVER, BC, V6B 1G1, CA

(73) Assignee: MIV Therapeutics Inc., Vancouver (CA)

(21) Appl. No.: 11/209,735

(22) Filed: Aug. 24, 2005

Related U.S. Application Data
(60) Provisional application No. 60/636,105, filed on Dec. 16, 2004.

(30) Foreign Application Priority Data
Jul. 8, 2005 (WO) PCT/CA05/01066

(51) Int. Cl. A61K 9/24

(52) U.S. Cl. 424/472

(57) ABSTRACT

This application relates to a multi-layer drug delivery device and a method of manufacture. The device comprises a substrate; at least one first layer on the substrate containing the drug and a first solvent; and at least one second layer applied to the first layer to regulate release of the drug from the first layer, wherein the second layer comprises a polymer. The first solvent substantially prevents direct contact between the drug and the polymer. When applied to the first layer, the polymer is preferably dissolved in a second solvent which is immiscible with the first solvent to substantially prevent inter-diffusion between the first and second layers. In one application the substrate is a medical device, such as an implantable stent, having a biocompatible outer surface. The second layer is preferably biodegradable, bioabsorbable and/or bioresolvable in vivo to permit gradual exposure of the first layer and elution of the drug therefrom.

### REFERENCE TO RELATED APPLICATION

This application claims priority from U.S. Provisional Patent Application Ser. No. 60/636,105 filed 16 Dec. 2004 and Patent Cooperation Treaty Application Serial No. PCT/CA2005/001066 filed 8 Jul. 2005.

### TECHNICAL FIELD

This application relates to a multi-layer drug delivery device and a method of manufacturing same.

### BACKGROUND

Drug-coated medical devices are well known in the prior art. For example, drug-eluting intravascular stents have been shown to improve overall therapeutic performance after implantation or deployment of a coated stent within the lesion of a blood vessel. Drugs such as paclitaxel are typically employed to reduce restenosis at the site of implantation.

In order to be effective, drug-eluting stents are engineered to carry and release drugs in a controlled manner. Conventional approaches involve incorporating a therapeutic drug in a polymer solution, then coating the stent with the polymer. Drug can then be released over a therapeutically effective period of time after deployment in vivo. For example, U.S. Pat. No. 6,585,764 issued 1 Jul. 2003 entitled Stent With Therapeutically Active Dosage of Rapamycin Coated Thereon describes delivery of the drug rapamycin via a polymer matrix as a drug carrier. The polymer includes both degradable and non-degradable components. The drug-polymer mixture is coated via spraying or dipping on to a stent to achieve controlled release of the drug.

Published United States patent application No. 2002016437 filed 7 Nov. 2002 entitled Polymeric Systems for Drug Delivery and Uses Thereof also exemplifies the prior art. This application describes mixing a single polymer or polymer blend with a drug to dissolve or suspend the drug within the blend.

Although many of the prior art drug delivery approaches of the prior art have been shown to be therapeutically effective, improved systems are desirable where the polymeric component is not used as a carrier for the drug, thus minimizing the amount of polymer required and improving the percentage of drug available for gradual elution. Further, improved systems are desirable where ordinarily water-insoluble drugs are delivered in a more soluble form at the target site.

### SUMMARY OF INVENTION

In accordance with the invention, a multi-layer drug delivery device is provided. The device includes a substrate; at least one first layer on the substrate containing the drug and a first solvent; and at least one second layer applied to the first layer to regulate release of the drug from the first layer, wherein the second layer comprises a polymer, and wherein the first solvent substantially prevents direct contact between the drug and the polymer.

The second layer is preferably biodegradable, bioabsorbable and/or bioresolvable so that the first layer is gradually exposed when the drug delivery device is deployed in vivo. In one embodiment, the drug delivery device may be a drug-eluting stent.

The second layer may be applied to the first layer as a polymer solution comprising the polymer dissolved in a second solvent. Preferably the first and second solvents are substantially immiscible to prevent inter-diffusion between the first and second layers. In one embodiment the first solvent is hydrophilic and the second solvent is hydrophobic. The first and second solvents may also have substantially different boiling points.

The first solvent may be selected from the group consisting of methanol, ethanol, ethylene glycol, propylene glycol, Cremorphor, DMSO, DENA, glycerol and mixtures containing two or more of the preceding solvents. The polymer may be selected from the group consisting of polylactide, polyglycolide, poly(lactide-co-glycolide), polycaprolactone, polysulfone and mixtures containing two or more of the preceding polymers. The second solvent may be selected from the group consisting of chloroform, methylene dichloride, methylene trichloride, ethylene dichloride, ethylene acetate, butyl acetate, hexanes, heptanes and mixtures containing two or more of such solvents.

In one embodiment the drug may be ordinarily insoluble or poorly soluble in water and may have anti-proliferative and/or anti-inflammatory properties. One example of a suitable drug is paclitaxel. The concentration of the drug in the first layer may be within the range of about 0.01% to 50% by weight.

The first layer may be applied to a biocompatible surface of the substrate, such as an outer oxide layer. In one embodiment, the device may include a plurality of first and second layers applied to the substrate. For example, the plurality of first and second layers may be applied in alternating layers. The identity, amount and/or dissolution rate of the drug present in at least some of the drug-containing first layers may differ from corresponding features of the drug present in at least some other of the first layers.

The invention also relates to a method of manufacturing a multi-layer drug delivery device as described above comprising providing a substrate; applying at least one first layer to the substrate, wherein the first layer comprises the drug dissolved in a first solvent; and applying at least one second layer to the first layer to regulate release of the drug from the first layer, wherein the second layer comprises a polymer dissolved in a second solvent. In accordance with the method, the first and second solvents are immiscible thereby preventing direct contact between the drug and the polymer.

The invention further relates to a method of controllably delivering a drug at a target location comprising providing a drug delivery device as described above; delivering the device to the target location; allowing the second layer to biodegrade, bioabsorb and/or bioresolve at said target location to expose the first layer; and releasing the drug from the first layer at the target location.

### BRIEF DESCRIPTION OF DRAWINGS

In drawings which illustrate embodiments of the invention, but which should not be construed as restricting the spirit or scope of the invention in any way,

FIG. 1 is a longitudinal sectional view of a multi-layer drug delivery vehicle applied to an implantable medical device.

FIG. 2 is a photograph showing the immiscibility in vitro of a highly hydrophobic PLGA solution and a highly hydrophilic paclitaxel-containing solution.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

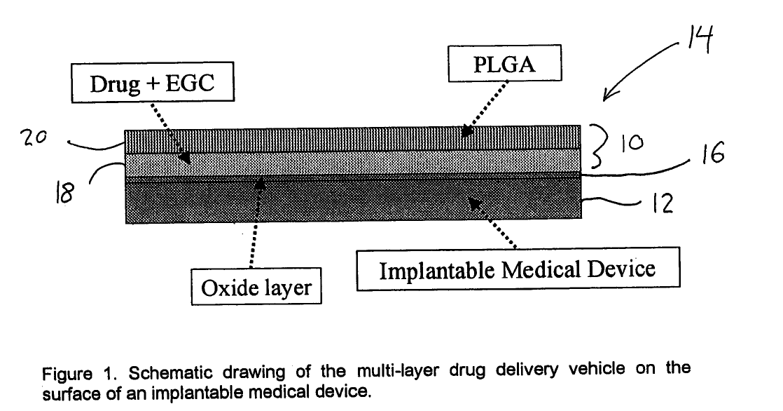

This invention describes a method for forming a multi-layer coating for drug delivery purposes. As shown in FIG. 1, the coating 10 is applied to a substrate 12 such as an implantable medical device. The resulting coated device is designated 14. Substrate 12 may optionally include some surface modification on its outer surface to which the coating 10 is applied. In the illustrated embodiment, an oxide layer 16 is applied to the outer surface of substrate 12. Oxide layer 16 may be formed, for example, by thermal or chemical means. As will be apparent to a person skilled in the art, other means of surface modification may be employed, such as the method described in Applicant's co-pending Patent Cooperation Treaty application No. PCT/CA2004/001585 which is hereby incorporated by reference in its entirety.

Although the present invention is described in relation to metal substrates such as implantable medical devices, the invention may be useful in other applications where it is desirable to deliver a drug to a target site. The invention may have application, for example, for use with medical devices which are not permanently implanted in vivo or medical devices used in peripheral rather than coronary applications.

As shown in FIG. 1, coating 10 includes an inner drug-containing layer 18 and an outer polymer-containing layer 20. As described in detail below, layers 18, 20 are substantially immiscible to prevent inter-diffusion between the layers 18,20 and, in particular, direct contact between the drug and the polymer. For example, in one embodiment described herein drug-containing layer 18 is highly hydrophilic and polymer-containing layer 20 is highly hydrophobic. In other possible alternative embodiments drug-containing layer 18 may be hydrophobic and polymer-containing layer 20 may be hydrophilic.

Although the coated medical device 14 of FIG. 1 includes a single multi-layer coating 10, it should be understood that alternative devices may contain multiple coatings 10. Moreover, each coating 10 may include more than drug-containing layer 18 and polymer-containing layer 20. However, within each coating 10 layers 18, 20 remain separate to prevent drug-polymer interaction as described above. In this invention the polymer is therefore not employed as a carrier for the drug.

In one embodiment of the invention, the drug containing layer 18 may be prepared as a hydrophilic solution. Although the invention is described in this embodiment as including the drug paclitaxel, other suitable drugs could be employed, including other ordinarily water-insoluble drugs. The hydrophilic drug-containing solution is formulated by dissolving a small amount of commercially-available paclitaxel into methanol or ethanol solvent under vigorously stirring until the paclitaxel is completely dissolved. In this particular example the resulting solution has a paclitaxel concentration of 1 to 6 weight percent. A small amount of ethylene glycol-Cremorphor mixture (hereinafter termed EGC), where the Cremorphor takes 0 to 20 weight percent in the EGC, is added into the paclitaxel-ethanol mixture.

The resulting paclitaxel-ethanol-EGC mixture can then be applied by via dipping, spraying or brushing on to substrate 12, such as a metal stent or other prosthesis. As mentioned above, substrate 12 may be pre-treated to form a thin oxide layer 16 on its outer surface, such as by thermal oxidation, sol-gel thin-film deposition, or chemical deposition methods known to the art. After the paxlitaxel-ethanol-EGC mixture is applied on to the outermost surface 16 of substrate 12, the volatile ethanol is rapidly removed under ambient temperature to yield a final film of paclitaxel-ethylene glycol mixture comprising the drug-containing layer 18. In this example, the final concentration of the paclitaxel in layer 18 is within the range of about 1 to 10 weight percent.

The polymer layer 20 is produced by first formulating a polymer containing solution. The polymer selected should be biodegradable, bioabsorbable and/or bioresolvable. The solvent used to dissolve the polymer should be immiscible with the solvent used to produce layer 18 as described above. For example, the polymer may include polylactide, polyglycolide, poly(lactide-co-glycolide), polycaprolactone, polysulfone and mixtures containing two or more of the preceding polymers in a methylene chloride solvent. Methylene chloride is a highly hydrophobic solvent which is immiscible with the EGC mixture described above. In one specific example, a solution of poly(lactide-co-glycolide)-methylene chloride is formulated. The concentration of PLGA in the solution is about 5 weight percent. The PLGA solution is dipped or sprayed coating onto the drug-containing layer 18. The methylene chloride solvent is then rapidly removed under vacuum or forced ventilation to form polymer layer 20. Layer 20 essentially functions as a protective topcoat on drug-containing layer 18 as described below.

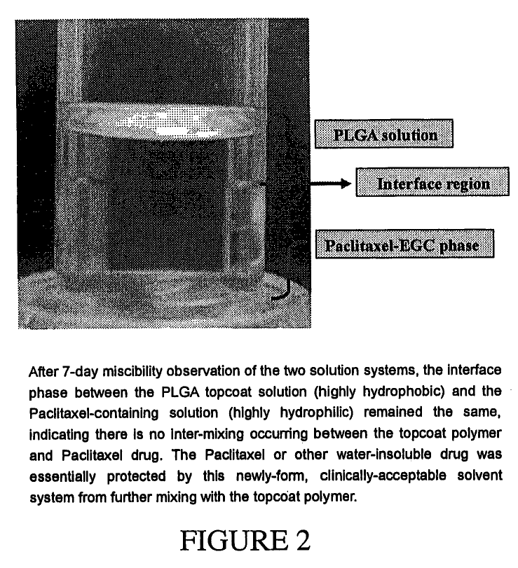

One key feature of this invention is that the high immiscibility of the hydrophilic ethylene glycol in layer 18 and hydrophobic methylene chloride in layer 20 prevents the underlying paclitaxel drug, which is readily being surrounded and protected by ethylene glycol molecules, from further dissolving in the methylene chloride to form a paclitaxel-PLGA mixture. Rather, layers 18, 20 forms a well-separated laminate comprising an underlying paclitaxel layer and PLGA topcoat barrier layer as shown in FIG. 1. The immiscibility of the polymer-containing solution and drug-containing solution is also shown in FIG. 2 which shows a clear separation between the solutions with no inter-mixing therebetween.

Another key feature of this invention is that the paclitaxel drug is well preserved in a dissolved configuration, rather than in a dried crystalline form, in the multi-layer coating, and an enhancement of water solubility due to the presence of said EGC by a factor of 2-3 orders was observed. The invention thus provides a new method of delivering paxlitaxel or other drugs via a novel multi-layer drug delivery vehicle.

### EXAMPLE

The examples contained herein illustrate the invention in further detail although it is appreciated the invention is not limited to the specific examples.

A 10% drug solution was initially prepared by dissolving commercially available paclitaxel in methanol solvent. A solvent mixture consisting of ethylene glycol, Cremophor and DMSO was then added to the drug solution to yield a final drug solution. The final drug solution was then applied to a metal substrate using a dip coating/spinning technique and the methanol solvent was removed. The drug and remaining solvent thus formed a first layer on the metal substrate consisting of a drug-containing paste.

A 5% polymer solution was prepared by dissolving PLGA in methylene chloride solvent. The resulting polymer solution was then applied to the first drug-containing layer using a dip coating/spinning technique. The methylene chloride solvent was allowed to evaporate under ambient conditions. The remaining polymer thus formed a protective second layer on the first drug-containing layer.

The coated substrate was placed in vitro in a dissolution apparatus containing phosphate buffered saline with 0.5% Tween-80. After 7 days the drug concentration in solution was measured using HPLC. The test confirmed the presence of paclitaxel, thus demonstrating the degradation of the outer polymer-containing second layer and elution of paclitaxel from the inner first layer.

The inventors have conducted animal studies of drug-eluting stents fabricated in accordance with the invention. The studies have shown that such stents exhibited very thin, uniform and complete endothelization and neovascularization in vivo without any apparent adverse affects.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

What is claimed is:

1. A multi-layer drug delivery device comprising:
(a) a substrate;
(b) at least one first layer on said substrate containing said drug and a first solvent, and
(c) at least one second layer applied to said first layer to regulate release of said drug from said first layer, wherein said second layer comprises a polymer,
wherein said first solvent substantially prevents direct contact between said drug and said polymer.

2. The device as defined in claim 1, wherein said second layer is biodegradable, bioabsorbable or bioresolvable.

3. The device as defined in claim 1, wherein said second layer is applied to said first layer as a polymer solution comprising said polymer dissolved in a second solvent, and wherein said first and second solvents are substantially immiscible.

4. The device as defined in claim 3, wherein said first solvent is hydrophilic and said second solvent is hydrophobic.

5. The device as defined in claim 3, wherein said first solvent is hydrophobic and said second solvent is hydrophilic.

6. The device as defined in claim 3, wherein said first solvent has a substantially different boiling point than said second solvent.

7. The device as defined in claim 1, wherein said first solvent is selected from the group consisting of methanol, ethanol, ethylene glycol, propylene glycol, Cremorphor, DMSO, DENA, glyerol and mixtures containing two or more of the preceding solvents.

8. The device as defined in claim 1, wherein said polymer is selected from the group consisting of polylactide, polyglycolide, poly(lactide-co-glycolide), polycaprolactone, polysulfone and mixtures containing two or more of the preceding polymers.

9. The device as defined in claim 3, wherein said second solvent is selected from the group consisting of chloroform, methylene dichloride, methylene trichloride, ethylene dichloride, ethylene acetate, butyl acetate, hexanes, heptanes and mixtures containing two or more of the preceding solvents.

10. The device as defined in claim 1, wherein said drug has anti-proliferative and/or anti-inflammatory properties.

11. The device as defined in claim 1, wherein said drug is ordinarily insoluble or poorly soluble in water.

12. The device as defined in claim 1, wherein said drug is paclitaxel.

13. The device as defined in claim 1, wherein the concentration of said drug in said first layer is within the range of about 0.01% to 50% by weight.

14. The device as defined in claim 1, wherein said first layer is applied to a biocompatible surface of said substrate.

15. The device as defined in claim 14, wherein said biocompatible surface comprises an oxide layer.

16. The device as defined in claim 1, comprising a plurality of said first and second layers applied to said substrate.

17. The device as defined in claim 16, wherein said plurality of first and second layers are applied in alternating layers.

18. The device as defined in claim 16, wherein the identity, amount and/or dissolution rate of said drug present in at least some of said first layers differs from corresponding features of said drug present in at least some other of said first layers.

19. The device as defined in claim 1, wherein said substrate is a metal.

20. The device as defined in claim 1, wherein said substrate is a medical device.

21. The device as defined in claim 20, wherein said substrate is a stent.

22. A method of manufacturing a multi-layer drug delivery device comprising:
(a) providing a substrate;
(b) applying at least one first layer to said substrate, wherein said first layer comprises said drug dissolved in a first solvent; and
(c) applying at least one second layer to said first layer to regulate release of said drug from said first layer, wherein said second layer comprises a polymer dissolved in a second solvent,
wherein said first and second solvents are immiscible thereby preventing direct contact between said drug and said polymer.

23. The method as defined in claim 22, wherein said substrate is biocompatible.

24. The method as defined in claim 22, wherein said second layer is biodegradable, bioabsorbable and/or bioresolvable.

25. The method as defined in claim 22, wherein said first solvent is hydrophilic and said second solvent is hydrophobic.

26. The method as defined in claim 22, wherein said first solvent is hydrophobic and said second solvent is hydrophilic.

27. The method as defined in claim 22, wherein said first solvent has a substantially different boiling point than said second solvent.

28. The method as defined in claim 22, wherein said first solvent is selected from the group consisting of methanol, ethanol, ethylene glycol, propylene glycol, Cremorphor, DMSO, DENA, glycerol and mixtures containing two or more of the preceding solvents.

29. The method as defined in claim 22, wherein said polymer is selected from the group consisting of polylactide, polyglycolide, poly(lactide-co-glycolide), polycaprolactone, polysulfone and mixtures containing two or more of the preceding polymers.

30. The method as defined in claim 22, wherein said second solvent is selected from the group consisting of chloroform, methylene dichloride, methylene trichloride, ethylene dichloride, ethylene acetate, butyl acetate, hexanes, heptanes and mixtures containing two or more of the preceding solvents.

31. The method as defined in claim 22, wherein said drug has anti-proliferative and/or anti-inflammatory properties.

32. The method as defined in claim. 22, wherein said drug is ordinarily insoluble or poorly soluble in water.

33. The method as defined in claim 22, wherein said drug is paclitaxel.

34. The method as defined in claim 22, wherein the concentration of said drug applied in said first layer is within the range of about 0.01 to 50% by weight.

35. The method as defined in claim 22, wherein said step of providing a biocompatible substrate comprises modifying an outer surface of said substrate.

36. The method as defined in claim 35, wherein said modifying step comprise forming an oxide layer on said outer surface by thermal oxidation, sol-gel thin film deposition or chemical pre-treatment deposition methods prior to application of said first layer.

37. The method as defined in claim 22, wherein applying at least one first layer to said substrate comprises:
(a) dissolving said drug in said first solvent to form a first solution;
(b) applying said first solution to said substrate; and
(c) removing said at least some of said first solvent from said first solution.

38. The method as defined in claim 35, wherein said first solvent comprises a mixture containing methanol and/or ethanol and said method comprises removing at least some of said methanol and/or ethanol after said first layer is applied to said substrate.

39. The method as defined in claim 37, wherein said first solution is applied to said substrate by dipping, spraying or brushing.

40. The method as defined in claim 22, wherein applying at least one second layer to said first layer comprises:
(a) dissolving said polymer in said second solvent to form a second solution;
(b) applying said second solution to said first layer; and
(c) removing said at least some of said second solvent from said second solution.

41. The method as defined in claim 40, wherein said second solvent comprises methylene chloride and said method comprises removing at least some of said methylene chloride after said second layer is applied to said first layer.

42. A method of controllably delivering a drug at a target location comprising:
(a) providing a drug delivery device as defined in claim 1;
(b) delivering said device to said target location;
(c) allowing said second layer to biodegrade, bioabsorb and/or bioresolve at said target location to expose said first layer; and
(d) releasing said drug from said first layer at said target location.

43. The method as defined in claim 42, wherein the step of allowing said second layer to biodegrade, bioabsorb and/or bioresolve at said target location is gradual.
